(19) United States
(12) Patent Application Publication
(10) Pub. No.: US 2016/0192828 A1
(43) Pub. Date: Jul. 7, 2016

(54) RETRACTOR CANNULA AND METHOD OF USE

(71) Applicant: Nicholes Sexton, Roseburg, OR (US)

(72) Inventor: Nicholes Sexton, Roseburg, OR (US)

(21) Appl. No.: 14/591,701

(22) Filed: Jan. 7, 2015

(51) Int. Cl. A61B 1/00; A61B 17/34

(52) U.S. Cl. CPC A61B 1/00154; A61B 17/3421

(57) ABSTRACT

A cannula capable of accommodating an endoscope, the cannula which includes at least one appendage extendable from the distal end of the cannula which can contact the underside of a layer of muscle or tissue when the cannula is in use, the cannula further adapted to releasably grip an endoscope inserted into it such that a surgeon can use the appendage to retract tissue by exerting a pulling force on an endoscope inserted through the cannula.

### BACKGROUND

Arthroscopy is a minimally invasive surgical technique in which examination and treatment of joint injuries are performed using instruments which are inserted through small incisions in the skin. In an arthroscopic procedure, an arthroscope is inserted into the joint through a small incision and used to visualize the joint. Instruments are inserted through different small incisions, and the joint repair is performed without the use of a large incision. Typically, cannulas are inserted into the small incisions, and instruments are inserted into joint via the cannulas. The arthroscope is typically not inserted through a cannula, but rather is encased in a sheath, and the sheathed arthroscope is inserted into the joint through an incision. The primary advantage of an arthroscopic procedure is the avoidance of cutting open a joint. The disadvantage of an arthroscopic procedure is that the surgeon must operate in limited space and with limited visibility.

To provide additional visibility, the patient's joint is typically distended with fluid which is pumped into the joint via portals in the sheath of the arthroscope. The distension of the joint creates space between tissues which permits the surgeon to see the tissues through the arthroscope. However, there are limits to how much space fluid pressure can create in a joint, and so space constraints typically remain. Additionally, traditional arthroscopes cannot be moved between different points of entry in a joint without a loss of fluid distension because fluid leaks from the joint through the various incisions, and a source of new fluid is necessary to maintain distension. When the scope and its sheath are removed from the joint so that they can be repositioned, the flow of fluid out of the incision that formerly held the arthroscope increases, and the source of fluid is removed. Fluid pressure can also suppress bleeding, and so a reduction in fluid pressure can lead to bleeding within the joint which further compromises visibility within the joint. To address these issues, Arthrex has developed sheathless arthroscopes, which are specially reinforced so that they do not require a sheath and can be inserted into the joint through cannulas which are ordinarily used to accommodate other instruments (normally referred to as “working cannulas”). Instead of being delivered through the arthroscope's sheath, fluid is delivered through one or more working cannulas. A sheathless arthroscope can be easily moved between working cannulas without loss of fluid pressure, more easily permitting multiple views of the joint.

While a substantial improvement, sheathless arthroscopic systems do not overcome the limited ability of fluid distension to create space within a joint. The surgeon still has only the amount of space that fluid pressure can create in which to work. Therefore, at least one conventional limitation on visibility in arthroscopic surgery remains. Moreover, this limitation is encountered in other types of surgical procedures in which endoscopes are used to visualize internal anatomy, such as laparoscopy, thorascopy, etc. The need remains for a method for enhancing operating space in the context of an endoscopic procedure.

### BRIEF DESCRIPTION OF THE DRAWINGS

FIG. 1 is a perspective view of a conventional cannula inserted into a body.

FIG. 2 is a perspective view of one embodiment of the inventive cannula disclosed herein inserted into a body.

FIG. 3 is a perspective view of one embodiment of the inventive cannula disclosed herein.

FIG. 3a is a perspective view of the end of the cannula depicted in FIG. 3.

FIG. 4 is a perspective view of an additional embodiment of the inventive cannula disclosed herein.

FIG. 4a is a perspective view of an additional embodiment of the inventive cannula disclosed herein.

FIG. 5 is an exploded view of one embodiment of the inventive cannula as disclosed herein.

FIG. 6a is a side view of a cannula.

FIG. 6b is a side view of a coupler as disclosed herein.

FIG. 6c is a top view of one embodiment of a coupler as disclosed herein.

FIG. 6d is a side view of a collar as disclosed herein.

FIG. 6e is a top view of a collar enclosing an elastomeric member as disclosed herein.

### DETAILED DESCRIPTION

Disclosed herein are various embodiments of a working cannula and mechanism for adapting a working cannula so that the cannula grips the endoscope. Gripping the endoscope means that the endoscope is removably affixed to the cannula, and moving the endoscope causes the cannula to move. The position of the endoscope can be adjusted within the cannula, but the endoscope is difficult to inadvertently dislodge. Further disclosed herein are various embodiments of a cannula which can be used to retract tissue when a user, such as a surgeon or an assistant, exerts a pulling force on it or on an instrument such as an endoscope inserted through it.

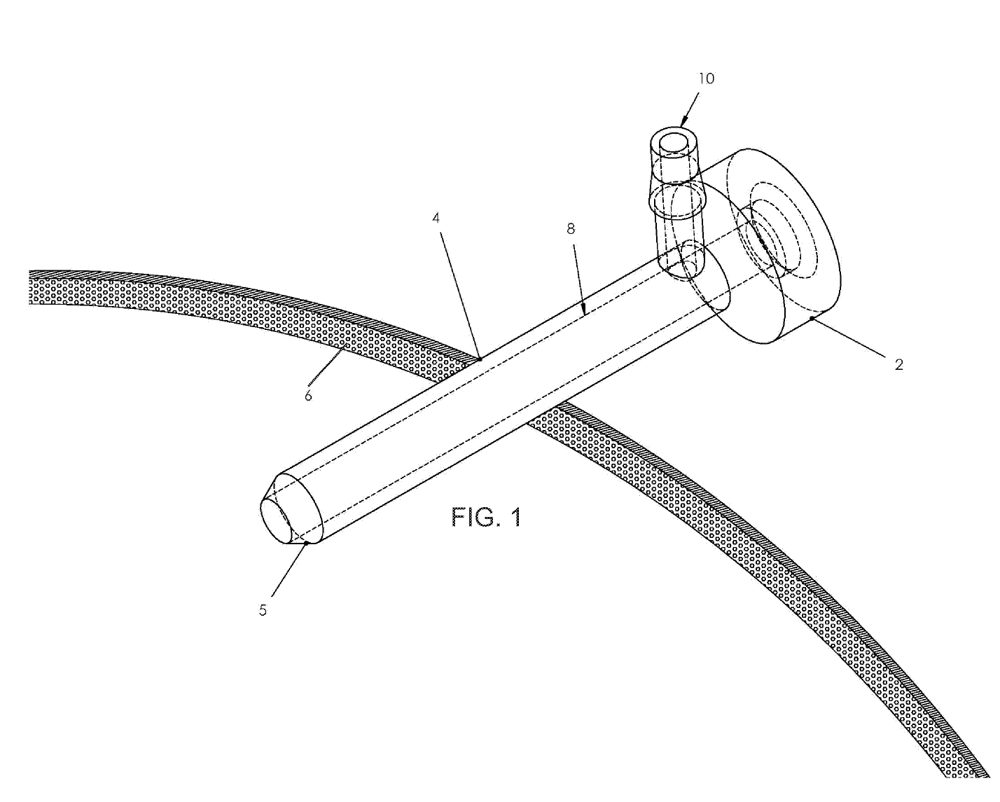

As shown in FIG. 1, cannula 2 is used to provide access to submuscular anatomy during surgery via an incision 4. A cannula 2 has a hollow shaft which defines a passage 8 which extends the length of the cannula and accommodates instruments or an endoscope inserted beneath a layer of muscle 6 or other tissue and into a patient's body. A cannula 2 may include a seal such as a through-hole seal made of silicone or an elastomer so that instruments can easily be passed into the cannula but fluid leakage is controlled. A cannula 2 may also include a port 10 for the injection of fluid into the joint in order to distend the joint.

During an arthroscopic surgery, cannulas are used to provide access to submuscular structures. Instruments such as obdurators, rasps, spears, drills, screwdrivers, and suturing instruments can be passed into the body of the patient, including into the joint, using cannulas. Because instruments are passed through the cannulas, they are sized so that they do not fit snugly around an instrument, but rather permit the instruments to move freely within the cannulas.

Endoscopes such as arthroscopes can also be inserted into the body of a patient and into joint spaces using cannulas, as described in US Patent Pub. No. 2013/0204083, the entirety of which is incorporated herein by reference. Endoscopes typically have integral cameras and light sources which enable the projection of images of internal structures including joint structures onto monitors so that the surgeon can visualize internal anatomy once the endoscope is inserted through the cannula and into the body. The same cannula maybe used to interchangeably accommodate instruments and an endoscope. The endoscope is inserted, the surgeon visualizes the joint, then the endoscope may be moved to a different cannula while an instrument is inserted into the first cannula in order to proceed with the operation. Endoscopes, like instruments, slide freely within a cannula.

Recently, Arthrex has introduced the Gemini cannula, which is described in U.S. Pat. No. 8,360,969, the entirety of which is incorporated herein by reference. The Gemini cannula has arms which can be deployed when the cannula is in place so that they extend outward from the distal end of the shaft of the cannula. The arms extend within the joint capsule, and maintain the position of the cannula by preventing the cannula from beneath the muscles and prevent the cannula from being inadvertently withdrawn when an instrument is withdrawn.

U.S. Pat. No. 8,360,969 discloses that the arms of the Gemini cannula can act as retractors as they move into the open position. However, surgeons do not typically manipulate cannulas once they are in place. Surgeons and surgical staff are typically fully occupied with manipulating instruments and other tasks. Surgical instruments must slide freely within cannulas so that the surgeon can change their positions within the joint in the course of their use. While sheathless arthroscopes can be inserted into a joint via a Gemini cannula, the arthroscope slides freely within the cannula so that a surgeon can position it during its use. A surgeon using a Gemini cannula cannot use it as a retractor by pulling on the instrument, but rather must let go of the instrument and devote a hand to pulling on the cannula. Moreover, the arms of the Gemini cannula are not big enough to have sufficient surface area in contact with tissues to reliably resist a deliberate outward pull sufficient to retract tissues and create more space in the joint. The designers of the Gemini cannula made the arms only large enough to resist the fairly minimal stresses exerted by the withdrawal of instruments. When it is subjected to a firm pull, the wings are often lost within or outside muscle fibers such as those in the deltoid because the wings lack sufficient surface area. Muscle layers are resilient structures, and a significant pull is necessary to retract them. Such a pull on a Gemini cannula would risk dislodging the cannula.

The inventor herein has observed that a surgeon will often maintain a grasp on the arthroscope throughout the procedure in the course of maintaining visualization of the joint. The surgeon has come to the unexpected realization that an arthroscope configured to act as a retractor or coupled to a cannula which is configured to act as a retractor would enable a surgeon to effectively create space within a joint to facilitate visibility in the ordinary course of manipulating the arthroscope to visualize the joint. What would also be desirable is a cannula which can accommodate an arthroscope as well as a variety of other instruments, but which can be adapted to selectively grip an arthroscope, and which is configured to permit the retraction of tissue when a surgeon exerts a pulling force on an arthroscope affixed within it. For example, such a cannula and scope can be used in the subacromial/bursal space beneath the deltoid muscle during repair of a rotator cuff tendon.

A cannula having appendages which can be extended after the cannula has been placed with its distal end within the body and which have fairly extensive surface area which to contact tissues when deployed. Such a cannula can be used to access the joint and to effectively retract tissues within the joint capsule with an outward pull by the surgeon in order to create more operating space and visibility without dislodging the cannula.

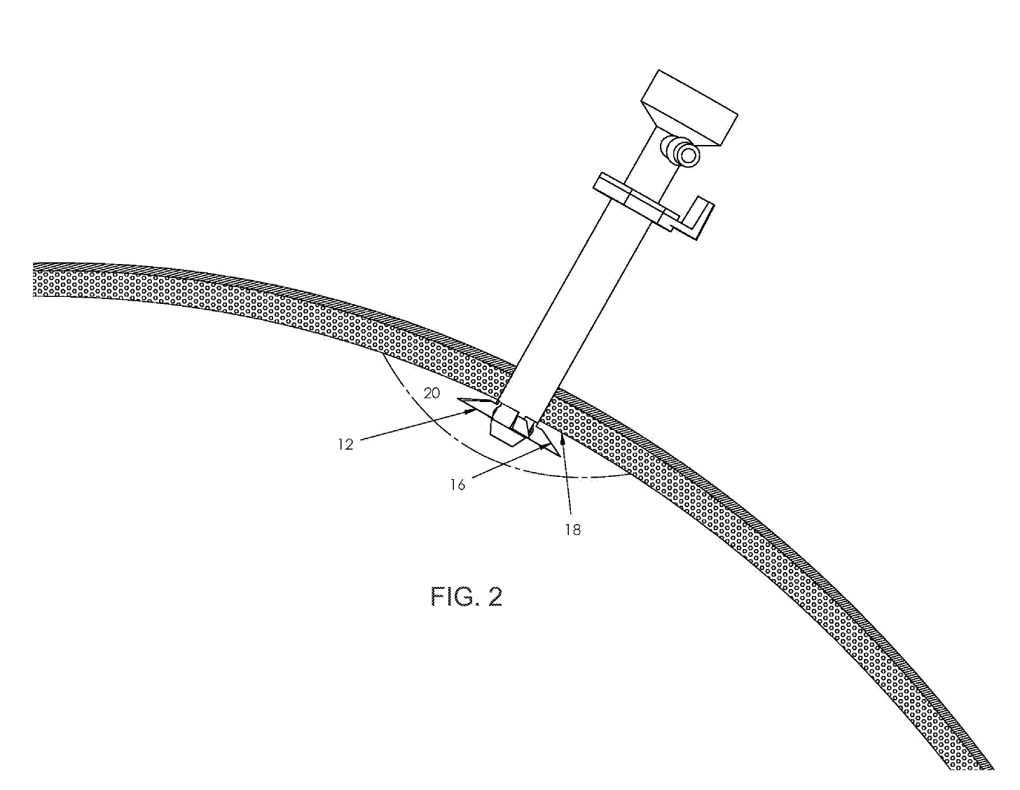

As shown in FIG. 2, cannula 2 extends through incision 4 so that its distal end 5 is beneath muscle layer 6. The cannula 2 defines an interior passage 8 to accommodate the passage of tools. Cannula may also include a fluid port 10 which defines a passage 11 used to feed fluid into the joint space in order to distend the joint. During use, instruments including a sheathless arthroscope are passed into the joint space through the cannula 2. Cannula 2 may also include a seal or membrane (not shown) in order to mitigate leakage of fluid out of the joint.

Cannula may include appendages 12 or wings which are deployable once the cannula is positioned with its distal end 5 in a patient's body. When in a pre-deployment position, the appendages 12 may converge to form a conical or frustoconical shape. When deployed, the appendages 12 open outward like the petals of a flower, and create surfaces 16 generally orthogonal to the long axis of the shaft of the cannula. These surfaces 16 contact the inner surfaces 18 of the musculature or other tissues such that when pulling force is exerted against them, they create space 20 within the joint or area of the body in which the operation is taking place.

The appendages may be approximately triangular, so that when they are in their pre-deployment position they come together to form a point in order to facilitate penetration through a small incision. They may be deployed by a variety of mechanisms. For example, U.S. Pat. No. 8,360,696, which is incorporated herein by reference in its entirety, discloses a cannula having arms which deploy and releasably lock in a deployed position due to the action of an internal cylinder, which slides telescopically within the outer cylinder of the cannula and pushes the arms into their deployed positions.

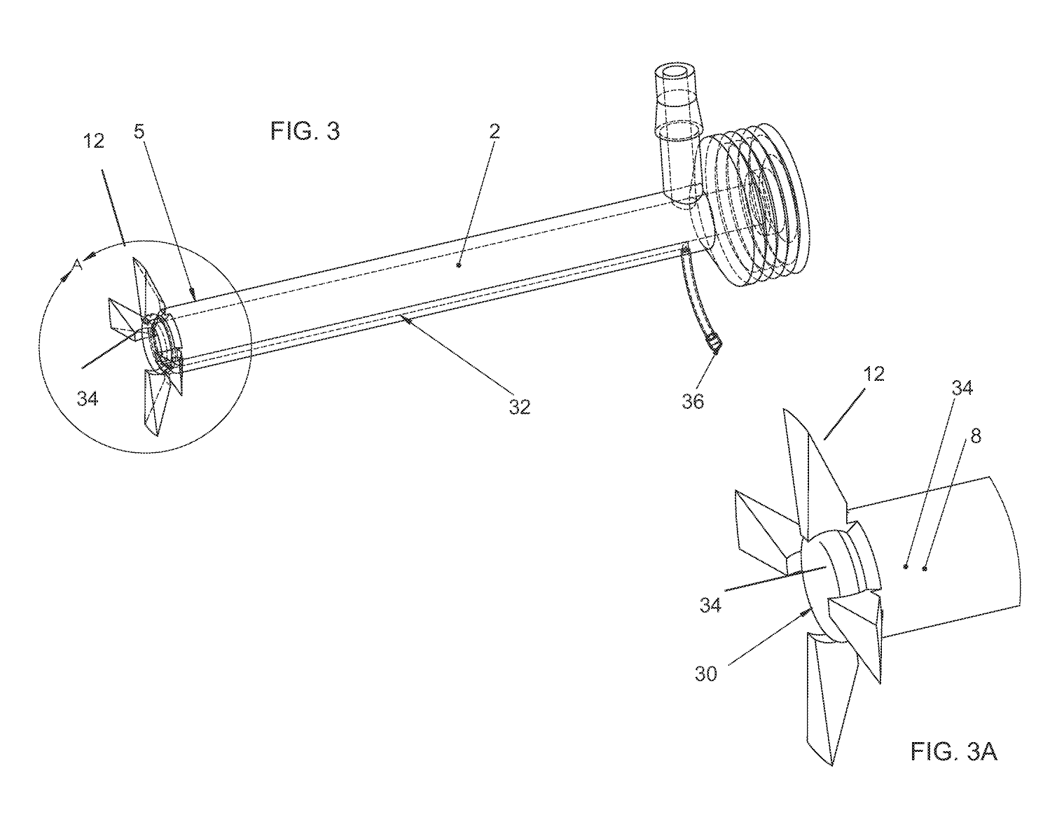

Alternatively, the appendages may be deployed due to the action of an inflatable bladder as shown in FIGS. 3 and 3a. The bladder 30 may be annular in shape and positioned at the distal end 5 of the cannula 2. When not deployed, the appendages12 may fold around the bladder 30. The bladder 30 may be interconnected with a tube 32 which follows the shaft of the cannula so that its terminal end 36 in a location outside the patient's body when the cannula is within use. Liquid or air is fed into the tube 32 and into the bladder 30, causing it to inflate or expand. When the bladder 30 inflates or expands as shown in FIG. 3a, it displaces the appendages 12 outward into a deployed position and maintains them in that position. Because the bladder 30 is annular, it defines an inner passage 34 which is aligned with the interior passage 8 of the cannula 2. When a scope or other instrument is fed through the cannula 2, the instrument or scope passes through the inner passage 34 of the bladder 30. The bladder may be sized so that it exerts some degree of compressive force on the instrument or scope. The degree of inflation or expansion of the bladder can be adjusted during the surgery by increasing or decreasing the amount of air or fluid in the bladder.

As disclosed in U.S. Pat. No. 8,360,969, such appendages could be integral with an external cannula, and can be extended by locking a telescoping internal cannula into an extended position. Alternatively, such an internal cannula may be engaged with the external cannula through threads, so that the position of the inner cannula with respect to the external cannula can be adjusted through screwing or unscrewing it.

Appendages may optionally be adjustable to achieve varying levels of retraction.

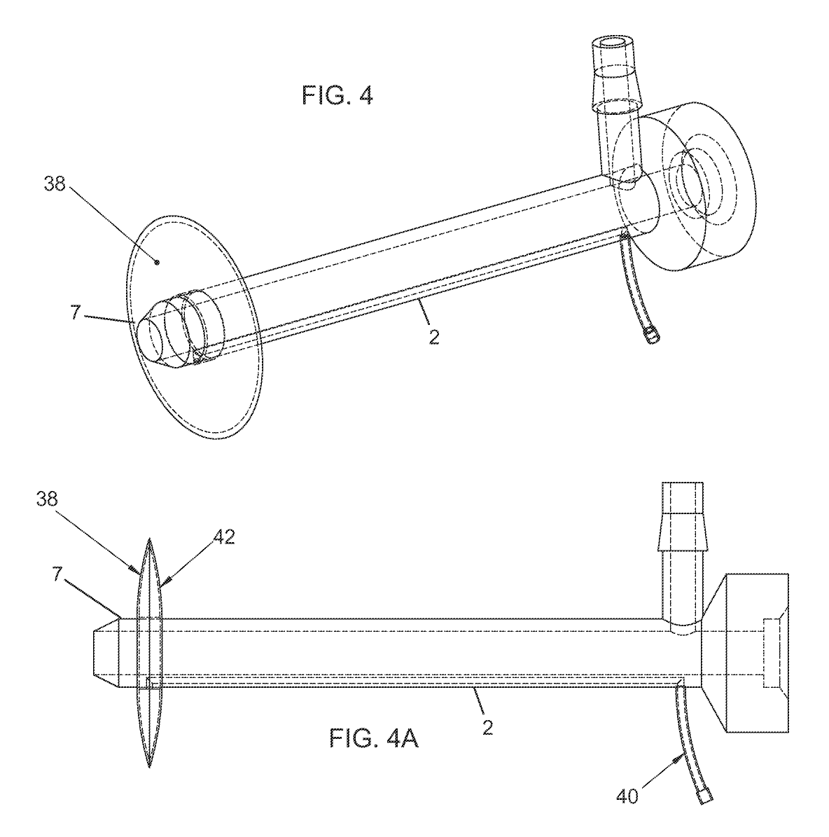

The cannula may additionally or alternatively include an annular inflatable wing 38 as shown in FIGS. 4 and 4a. The annular inflatable wing 38 can be selectively inflated with air or fluid through a tube 40 as discussed with reference to the bladder 30. When the cannula is in place, the wing can be inflated and will create a surface 42 which can be used to exert pressure on the tissues or joint capsule so that when a pulling force is exerted against the cannula it retracts the tissues. The inflatable wing will preferably be relatively thin so that it does not occupy too much space. As with the bladder discussed above, a syringe or small fingertip pump and release valve (not shown) could be utilized to inflate or deflate. The wing could alternatively be positionable in a variety of places along the sheath of a conventional scope or along a cannula.

Cannulas in accordance with some aspects of the invention described herein are adapted to grip an arthroscope inserted through them. This grip should be releasable and adjustable, but it should permit a surgeon exert a pulling force on the cannula by exerting a pulling force on the arthroscope sufficient to cause the appendages to retract the tissues and create more space within the joint or other area of the body being operated upon.

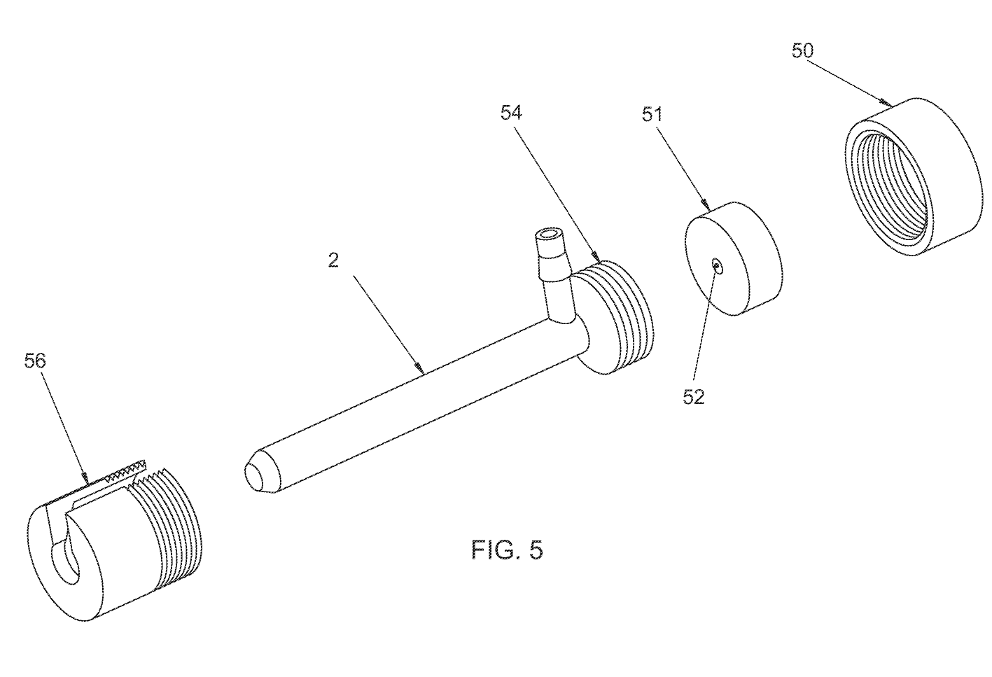

Referring to FIG. 5, in one embodiment, a collar 50 is removably affixed to the cannula 2. The collar is annular, and encloses or is integral with an annular elastomeric member 51 which defines an inner diameter 52 which may be slightly smaller than the cross section of a typical arthroscope. When the arthroscope is inserted into the cannula, it is inserted through the collar and through the elastomeric member. Pressure applied to the elastomeric member, for example by the collar, deforms the elastomeric member and decreases the size of internal diameter 52, enabling the elastomeric member to seal around and exert a compressive force upon the arthroscope even if inner diameter is not otherwise smaller than the arthroscope. The compressive force permits the arthroscope and attached cannula to be used as a retractor.

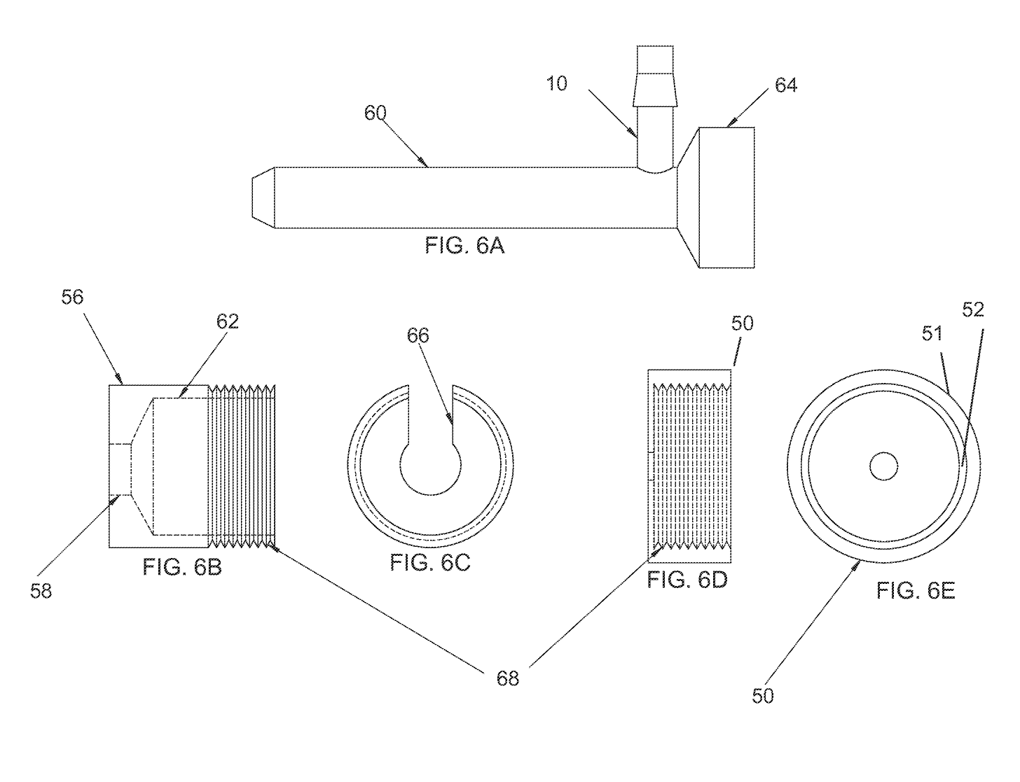

As shown in FIGS. 4 and 6a-6f, the collar 50 can be screwed onto a threaded portion 54 of the cannula 2. Alternatively, a removable coupler 56 can be used with a cannula which does not contain a threaded portion. The coupler is sized and shaped to fit over the cannula 2. Coupler may have a narrow portion 58 shaped to fit closely around the shaft 60 of the cannula, and a wider portion 62 shaped to accommodate the wider port 64 at the proximal end of the cannula. The coupler may also include a notch 66 which accommodates the fluid port 10 integral with the cannula 2. The coupler 56 includes a mechanism for attachment to the collar 50, such as threads 68 or other such known mechanisms. The coupler 56 and the collar 50 encase the elastomeric member 51, which exerts a compressive force on an arthroscope inserted through it permitting it to be used as a retractor, as discussed above.

As also discussed above, an annular inflatable bladder 30 positioned at the tip of the cannula may also exert sufficient compressive force on an arthroscope to permit use of the cannula and arthroscope as a refractor. Additionally or alternatively to the elastomeric member, a variety of releasable clamps known in the art and used conventionally for other purposes may be used to affix the cannula to the arthroscope.

What is claimed is:

1. A cannula for permitting passage of an endoscope into a body of a patient, said cannula comprising:
a shaft having a proximal end and a distal end, said shaft defining a passage,
one or more appendages affixed to said distal end of said shaft capable of being selectively extended in a direction which is generally orthogonal to said shaft, and
an elastomeric member affixed to said cannula, said elastomeric member defining an interior passage sized to exert a compressive force around said endoscope when said endoscope is inserted through said cannula.

2. The cannula of claim 1 wherein said elastomeric member is affixed near said proximal end of said cannula.

3. The cannula of claim 1 wherein said one or more appendages may be at least partially filled with air or water.

4. The cannula of claim 1 wherein said elastomeric member is at least partially encased in a threaded member.

5. The cannula of claim 1 wherein said elastomeric member is approximately annular.

6. The cannula of claim 1 wherein said elastomeric member is releasably affixed to said cannula.

7. A method of retracting tissue during surgery comprising:
providing a cannula, said cannula comprising a shaft having a proximal end and a distal end, said shaft defining a passage, one or more appendages affixed to said distal end capable of being selectively extended in a direction which is generally orthogonal to said shaft of said cannula, and an elastomeric member affixed to said cannula, said elastomeric member defining an interior passage sized to exert a compressive force around said endoscope when said endoscope is inserted through said cannula;
inserting said cannula through an incision in said patient's skin and at least one muscle layer such that said distal end extends through said muscle layer;
causing said one or more appendages to extend;
inserting said endoscope through said cannula; and
exerting a pulling force on said endoscope.

8. The method of claim 7 wherein said elastomeric member is affixed near said proximal end of said cannula.

9. The method of claim 7 wherein said one or more appendages may be at least partially filled with air or water.

10. The method of claim 7 wherein said elastomeric member is at least partially encased in a threaded member.

11. The method of claim 7 wherein said elastomeric member is approximately annular.

12. The method of claim 7 wherein said elastomeric member is releasably affixed to said cannula.
